(19) United States
(12) Patent Application Publication
(10) Pub. No.: US 2024/0390555 A1
(43) Pub. Date: Nov. 28, 2024

(54) METHOD OF PRODUCING THREE DIMENSIONAL AUTOLOGOUS FAT GRAFT USING HUMAN LIPOASPIRATE-DERIVED ADIPOSE TISSUE WITH MULTIPOTENT STEM CELLS AND BIOCOMPATIBLE CELLULOSE NANOFIBRILS

(71) Applicant: CELLHEAL AS, Sandvika (NO)

(72) Inventor: Paul Gatenholm, Riner, VA (US)

(73) Assignee: Ocean Tunicell AS, Blomsterdalen, OT (NO)

(21) Appl. No.: 17/268,924

(22) PCT Filed: Aug. 16, 2019
(86) PCT No.: PCT/IB2019/000922

§ 371 Date: Feb. 16, 2021

Related U.S. Application Data
(60) Provisional application No. 62/719,162, filed on Aug. 17, 2018.

(51) Int. Cl. A61L 27/38; A61L 27/20; A61L 27/26; A61L 27/36; A61L 27/56

(52) U.S. Cl. CPC A61L 27/3834; A61L 27/20; A61L 27/26; A61L 27/3604; A61L 27/56; A61L 2400/06; A61L 2430/34; A61L 2430/40

(57) ABSTRACT

Autologous 3D fat grafting and modification of adipose tissue lipoaspirate rich in stem cells with biocompatible cellulose nanofibrils (CNF) or collagen nanofibrils to achieve processable dispersion/emulsion which can be formed into a 3D shape(s) with desired porosity. 3D construct formed is further crosslinked either in situ at the implantation site or before implantation. Grafting can be performed in human or animal to correct soft tissue contour deformities in reconstructive and aesthetic surgery, including but not limited to injection, moulding, or 3D bioprinting. Biocompatible cellulose or collagen nanofibrils act as a dispersant/emulsifier of disintegrated lipoaspirate and provide shear thinning properties which make it possible to process dispersion into 3D shape yet provide porosity for nutrient diffusion and neovascularization. Invention further comprises a crosslinker(s) to provide construct's mechanical stability and ability to be transplanted as larger structure.

### CROSS-REFERENCE TO RELATED APPLICATIONS

The present application relies on the disclosures of and claims priority to and the benefit of the filing date of U.S. Provisional Application No. 62/719,162, filed Aug. 17, 2018. The disclosures of that application are hereby incorporated by reference herein in their entireties.

### BACKGROUND OF THE INVENTION

### Field of the Invention

The present invention generally relates to autologous fat grafting and more particularly relates to a method of producing three dimensional autologous fat graft using human lipoaspirate-derived adipose tissue, including multipotent stem cells and biocompatible cellulose nanofibrils.

### Description of Related Art

Autologous fat grafting is an established reconstructive and aesthetic surgical procedure because of the abundance of fat tissue and the relative simplicity of the procedure. The applications of fat grafting include facial rejuvenation, hand rejuvenation, breast reconstruction and volume enhancement, treatment of skin photoaging, correction of contour deformities, and improvement of senile and diabetic plantar fat pad atrophy. The process typically consists of three steps: harvesting of adipose tissue from a suitable donor site: processing of the lipoaspirate: and reinjection of the purified adipose tissue. The outcome is, however, poor and unpredictable. The main problem after autologous adipose tissue transplantation is its absorption rate over time, which results in an average reduction from 25% to 70% of the total implanted volume (see, Simonacci, F., Bertozzi, N., Grieco, M. P., Grignaffini, E. and Raposio, E., Procedure, applications, and outcomes of autologous fat grafting. Annals of Medicine and Surgery 20, 49-60 (2017)). Adipose cells die and become resorbed and the empty space allow infiltration of macrophages and polynucleated cells for phagocytosis. Grafted adipocytes pass through an ischemic phase. Vascularization has been observed when the fat fragments are very small.

Further, fat tissue is highly vascularized and contains several types of cells in addition to adipocytes. There is a population of pericytes close to a vascular network, endothelial progenitor cells, and adipose stem cells which are mesenchymal stem cells. The isolation of Stromal Vascular Fraction (SVF) from lipoaspirate is possible through digestion of an extracellular matrix along with several enzymes of which collagenase is typically the most important. SVF can be isolated by a manual procedure or by an automated procedure using commercially available automation equipment which enzymatically digest extracellular matrix, and wash and separate (examples include: Cytori Celution 800/CRS System, Medi-Khan's Lipokit and Icellator from Tissue Genesis). Adipose derived Stem cells (ASC) can then be plated out from SVF fraction and further expanded. SVF has been recognized as a potent cell fraction with the capability of healing and promoting vascularization. Isolation of SVF is also, however, associated with use of digestive enzymes which might have adverse effects for patients, and, at the time of this application, the regulatory approval is still pending. Other procedures of fat disintegration without use of enzymes include mechanical treatment, which is applied for example in a device from Lipogems, Italy (see Bianchi, F. et al., A new nonenzymatic method and device to obtain a fat tissue derivative highly enriched in pericyte-like elements by mild mechanical forces from human lipoaspirates. Cell Transplant. 22, 2063-2077 (2013): Tremolada, C., Colombo, V. & Ventura, C., Adipose tissue and mesenchymal stem cells: state of the art and Lipogems® technology development. Current stem cell reports 2, 304-312 (2016)).

A challenge met by the current invention is to improve survival of a fat graft, which, if can be achieved, results in the autologous fat grafting, which can be effectively used for wound healing and reconstructive and aesthetic surgery.

According to the invention herein, a possible way to improve survival of a fat graft would be to encapsulate it in a biopolymer matrix in such a pattern to create porosity providing for the ability of diffusion of nutrients and oxygen, as well as facilitating vascularization. Several biopolymers have been used as scaffolds and bioinks for general tissue engineering (see, e.g., Domingues, R. M. A., et al., Development of Injectable Hyaluronic Acid/Cellulose Nanocrystals Bionanocomposite Hydrogels for Tissue Engineering Applications, Bioconjugate Chem., 2015, 26 (8), pp. 1571-1581: Gatenholm, P., Cellulose nanofibrillar bioink for 3d bioprinting for cell culturing, tissue engineering and regenerative medicine applications, Patent Application, WO2016100856A1) wherein the biomaterials are combined with single cells. Polymer solutions are shear thinning, which means that the viscosity is decreased with increased shear rate. Cellulose powder composed of cellulose nanocrystals has been proposed for use in tissue engineering as described in patent application WO2007066222 A1, but such material does not provide sufficient nanofibrillar architecture.

Cellulose nanofibrils (CNF), which can be isolated from tunicates, produced by bacteria or isolated from primary or secondary cell walls of plants, are 8-30 nm in diameter and can be up to a micrometer long. They have a hydrophilic surface and therefore bind water on their surfaces forming hydrogels already at low solid content (1-2%). CNF are shear thinning and have high zero shear viscosity. The hydrophilic nature of the CNF surfaces covered by water prevent them from protein adsorption and make them bioinert. Cells do not recognize CNF surfaces which are an advantage, as taught herein, when it comes to biocompatibility since there is little to no foreign body reaction. Among various cellulose nanofibrils, tunicate derived nanofibrils have high crystallinity and thick fibril dimension with fibril lengths that can exceed 1 micrometer. According to the invention herein, this plays a role in dispersing power and viscoelastic properties. Nanocellulose fibrils combined with crosslinked alginate or hyaluronate have been shown to act as a suitable scaffold for differentiation of adipose progenitor cells (see, e.g., Henriksson, I., P. Gatenholm, and D. A. Hagg, Increased lipid accumulation and adipogenic gene expression of adipocytes in 3D bioprinted nanocellulose scaffolds. Biofabrication. 2017 Feb. 21: 9 (1): Krontiras, P., P. Gatenholm, and D. A. Hagg, Adipogenic differentiation of stem cells in three-dimensional porous bacterial nanocellulose scaffolds. Journal of Biomedical Materials Research Part B-Applied Biomaterials, 2015. 103 (1): p. 195-203) but not in combination with adipose cells.

### SUMMARY OF THE INVENTION

The invention herein overcomes the above challenges by introducing a method of producing a three dimensional autologous fat graft using human lipoaspirate-derived adipose tissue with multipotent stem cells and biocompatible cellulose nanofibrils. In a preferred embodiment, the three dimensional autologous fat graft using human lipoaspirate—derived adipose tissue is rich in or comprises a high amount or content of multipotent stem cells and biocompatible cellulose nanofibrils.

One embodiment herein provides a method of producing three dimensional autologous fat graft(s) with porosity, which survives implantation without volumetric shrinkage and becomes neovascularized to be used in aesthetic and reconstructive surgery in which the lipoaspirate, preferably rich in stem cells, is mixed with one or more excipients and/or biocompatible material(s) comprising nanofibrillar cellulose derived from tunicates, bacteria or plants, and crosslinked.

Another embodiment provides a method of producing three dimensional autologous fat graft(s) with porosity, which survives implantation without volumetric shrinkage and becomes neovascularized to be used in aesthetic and reconstructive surgery in which the lipoaspirate, preferably rich in stem cells, is mixed with one or more biocompatible material(s) comprising nanofibrillar cellulose derived from tunicates, bacteria or plants, and crosslinked.

In another embodiment, the invention herein comprises a method of producing a three dimensional autologous fat graft with porosity, which survives implantation without volumetric shrinkage and becomes neovascularized, to be used in aesthetic and reconstructive surgery, in which lipoaspirate, preferably rich in stem cells, is mixed with one or more excipient and/or one or more biocompatible material(s) comprising nanofibrillar cellulose derived from tunicates, which is crosslinked. In aspects, the nanofibrillar cellulose derived from tunicates is in the form of dispersion with fibrils of a diameter above 15 nm and length between 0.5 microns and 5 microns. In another embodiment, the nanofibrillar cellulose is derived from tunicates in the form of dispersion with solid content higher than 1.5% and lower than 4% by weight. The nanofibrillar cellulose derived from tunicates, in aspects, has no detectable hemicelluloses that affects immunoresponse and little to no detectable agglomerates. In an embodiment, nanofibrillar cellulose derived from tunicates is in the form of dispersion with no little to no detectable bacteria derived lipopolysaccharides known in cases as endotoxins.

In another embodiment, the nanofibrillar cellulose derived from tunicates is crosslinked by addition of alginate and Calcium, Barium and/or Strontium ions. In another embodiment, nanofibrillar cellulose derived from tunicates is crosslinked by addition of alginate and polylysine. In another embodiment, the nanofibrillar cellulose derived from tunicates is crosslinked by addition of a solution fibrinogen and/or thrombin.

In an embodiment, the nanofibrillar cellulose derived from tunicates is crosslinked by addition of platelet rich plasma (PRP) followed by or preceded by addition of Calcium ions. In another embodiment, the nanofibrillar cellulose derived from tunicates is crosslinked by addition of riboflavin 5′-phosphate followed by or preceded by exposure to UV (ultraviolet) light.

Another embodiment comprises an implantable tissue prepared for use in aesthetic and reconstructive surgery of a human. In an embodiment, an implantable tissue is prepared for use in aesthetic and reconstructive surgery of animals, including humans or other animals. In another embodiment, an implantable tissue is prepared for wound healing. In another embodiment, implantable biomaterial is prepared without addition of lipoaspirate and is used as a dermal filler.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The accompanying drawings illustrate certain aspects of embodiments of the present invention and should not be used to limit the invention. Together with the written description the drawings explain certain principles of the invention.

FIGS. 1A-C show various steps of lipoaspirate collection and processing techniques.

FIG. 2 shows rheological properties of disintegrated lipoaspirate (L), mixed with alginate (LA), mixed with alginate and nanocellulose from tunicates (LT).

FIG. 3 shows crosslinking kinetics measurements.

FIGS. 4A-B show results from 3D shaping experiments.

FIGS. 5A-D show results from in vivo experiments.

### DETAILED DESCRIPTION OF VARIOUS EMBODIMENTS

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein comprise a method based on mixing mechanically disintegrated lipoaspirate, enriched with stem cells, with biopolymers to achieve partially or fully homogenous dispersion, and then processing the mixture/using the mixture to create three dimensional architecture with porosity. The biopolymer materials, in aspects, are used as a dispersant/emulsifier. Porosity according to the current invention enables diffusion of nutrients and/or oxygen and contributes to survival of adipose cells, but it also allows for differentiation of progenitor cells and neovascularization. Grafting according to the present invention can be implemented by techniques, including but not limited to moulding, 3D Bioprinting, or injection into a human or animal body. Biopolymers described herein have, in aspects two different functions: first, providing homogeneous dispersion with shear thinning properties (pseudoplastic), and second crosslinking. Homogeneous dispersion/emulsion is achieved by adding biocompatible cellulose nanofibrils and/or other excipient(s), and crosslinking is achieved for example by using fibrinogen and thrombin or alginate and calcium, addition of platelet rich plasma (PRP) followed by addition of Calcium ions, and/or addition of riboflavin 5′-phosphate followed by exposure to UV light. Cellulose nanofibrils, particularly isolated from tunicates, in aspects, are preferred as dispersing and emulsifying agent(s), but they may also provide shear thinning properties, high viscosity at low shear stresses, and mechanical stability of the 3D fat graft. They also may enable improved porosity, which may result in neovascularization or improved neovascularization. Such described 3D autologous fat graft, in a preferred embodiment, survives implantation and does not volumetrically shrink to a medical meaningfully degree. It also gets vascularized in a preferred embodiment. The invention in this manner provides an innovative way to heal wounds and enable corrections and repair of soft tissue in reconstructive and aesthetic surgery.

### Example 1 (Lipoaspirate Collection and Processing)

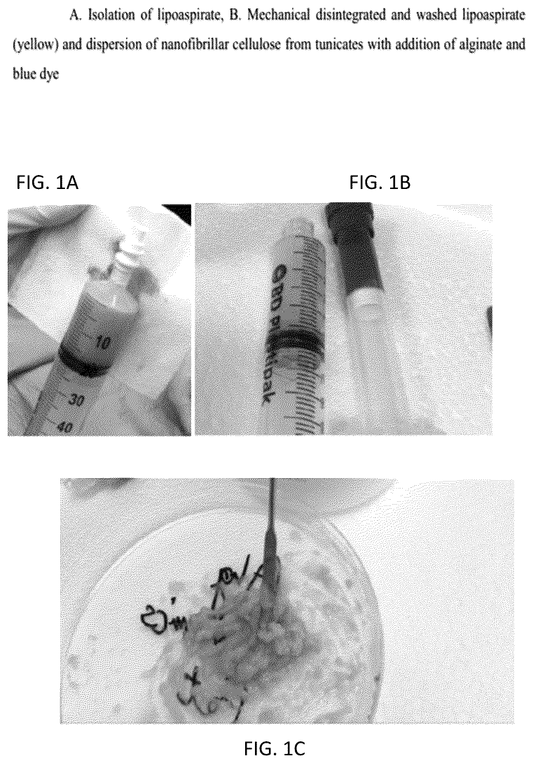

Liposuction aspirate was collected from the abdominal subcutaneous tissue of a healthy female donor with approval from the Regional Ethics Committee of Gothenburg (Dnr 624-16) and after signing informed consent. Lipoaspirate was processed with a Lipogems device, Italy, according to the manufacturer's protocol by mechanical force and without any enzymatic treatment. Briefly, lipoaspirate was washed with a physiologic electrolyte solution (Ringer's Acetate, Braun), emulsified by shaking with metal beads 5×20s, and rinsed in a closed system. A total of 160 ml of lipoaspirate was used from the donor. The processed lipoaspirate-derived adipose tissue described previously was mixed with 3% by weight solution of alginate SLG100 (Novamatrix, Norway) in 4.5% mannitol solution at a volumetric ratio of 90:10. The adipose mixture was further mixed with nanocellulose/alginate (80:20) in a 1:1 ratio. FIG. 1A shows the isolated lipoaspirate and FIG. 1B shows the processed form with Lipogems device lipoaspirate (yellow) and dispersion of cellulose nanofibrils from tunicates with added alginate and blue dye (blue). Figure IC shows green homogenous dispersion with paste-like consistency when processed lipoaspirate was mixed with cellulose nanofibril dispersion.

### Example 2 (Rheology)

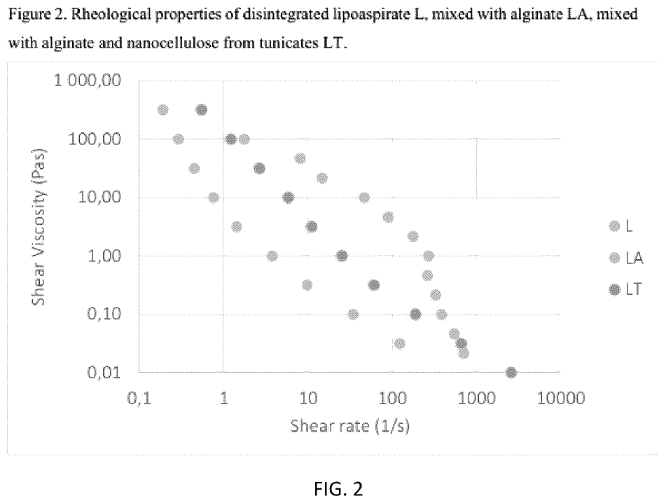

The rheological properties and crosslinking kinetics of the lipoaspirate-derived adipose disintegrated tissue prepared by the Lipogems system (L), and the processed adipose tissue mixed with alginate (LA) and alginate and nanocellulose from tunicates (LT), were analysed using a Discovery HR-2 rheometer (TA Instruments, UK) equipped with a peltier plate. All measurements were conducted at 25° C. Shear viscosity was measured at shear rates from 0.01-1500 s-1 with a plate-plate geometry (20 mm, gap=500 μm). The mechanically processed lipoaspirate-derived adipose tissue (L) was inhomogeneous and consisted of clusters of adipose tissue and free flowing liquid. The viscosity curve for L was non-linear due to the inhomogeneity of the material (see FIG. 2, upper curve). Addition of alginate provided more homogeneous dispersion with shear thinning properties (see lowest curve in FIG. 2). Viscosity was, however, too low for optimal 3D shaping. Addition of tunicate derived cellulose nanofibrils resulted in shear thinning properties and viscosity allowing 3D shaping (see curve LT in FIG. 2).

### Example 3 (Crosslinking)

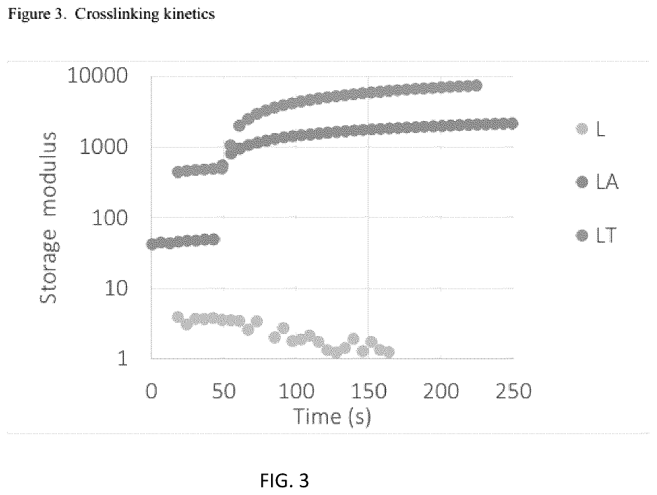

The crosslinking experiments were performed to evaluate different crosslinkers and also kinetics of crosslinking. FIG. 3 shows results from experiments when alginate was used and crosslinking was performed with addition of 0.1 M CaCl₂ solution. Oscillation frequency measurements were conducted at stresses within the measured LVR region and at a frequency range of 0.1-100 rad/s. Oscillation time measurements were conducted at 1.5% strain and a frequency of 1 Hz for 10 minutes. 20 seconds after initiating the measurement, 1 ml of 0.1 M CaCl2 was dispensed around the measured lipoaspirate dispersions while collecting data on the storage modulus. The lowest curve in FIG. 3 was lipoaspirate (L) and it was not crosslinked by addition of CaCl₂ solution. In contrast, both lipoaspirate with alginate (LA) and with alginate and cellulose nanofibrils isolated from tunicates were crosslinked within 60-100 seconds, as seen by the quick increase in moduli upon addition of CaCl₂ at t=40s. It can be seen in FIG. 3 that the storage modulus of crosslinked lipoaspirate is higher when cellulose nanofibrils from tunicates are added compared only with alginates. Additional crosslinking experiments were performed, wherein instead of an alginate and CaCl₂ solution, a fibrinogen and thrombin solution was added. The dispersion crosslinked fast after the thrombin addition. Addition of platelet rich plasma (PRP) followed by addition of Calcium ions also provided crosslinking. Addition of riboflavin 5′-phosphate followed by exposure to UV was also performed and resulted in crosslinking.

### Example 4 (3D Shaping: Moulding and 3D Bioprinting)

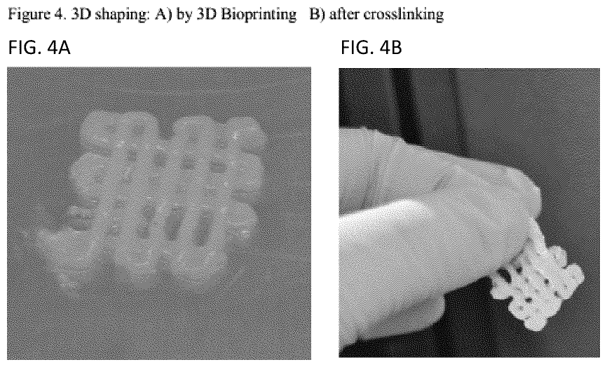

3D shaping ability of the lipoaspirate-derived adipose tissue processed by the Lipogems system solely (L), or mixed with alginate (LA), and alginate and nanocellulose (LT). respectively. was evaluated by moulding and printing with the pneumatic extrusion based 3D bioprinter “Inkredible” (Cellink AB. Sweden). Grid designs were printed with a conical nozzle (diameter 800 micrometer). The ability to dispense the material continuously through the nozzle and obtain printed structures with a printing fidelity for which the grid design was visible was evaluated. Neither lipoaspirate alone (L) nor with alginate (LA) was optimally able to be shaped into 3D structures. Only lipoaspirate with addition of cellulose nanofibrils derived from tunicates was optimally processed/formed into 3D structures, as can be seen in FIG. 4. FIG. 4A shows the result of grid architecture before crosslinking, and FIG. 4B shows results after crosslinking. The 3D structure can be lifted and implanted in a preferred embodiment.

### Example 5 (Implantation of 3D Grafts)

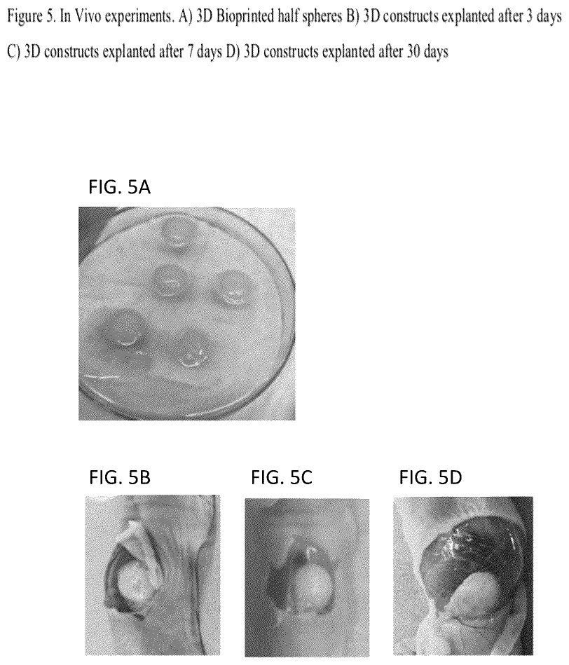

For in vivo experiments. the half spheres (diameter of 10 mm, height of 3 mm) were 3D Bioprinted. crosslinked with CaCl₂ for 5 minutes and thereafter immediately implanted subcutaneously in BALB/c nude mice. FIG. 5A shows 3D Bioprinted half spheres as they were 3D Bioprinted and crosslinked. FIG. 5B shows 3D constructs in mice after 3 days; the 3D shape of the implanted half sphere has mostly not changed. FIG. 5C shows 3D constructs after 7 days of implantation; there is mostly no change in volume of the construct and it appears as if it has become more elastic. Some vascularization was observed on the surface of explant. FIG. 5D shows 3D constructs after 30 days implantation; there is little to no volume contraction. Opposite, the construct seems to increase slightly in volume. become elastic, change colour into more yellow, and partially or fully vascularize.

The foregoing description of the specific embodiments will so fully reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compounds and/or compositions disclosed herein or with any other compounds and/or compositions. Likewise, any of the disclosed compounds and/or compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range, to the tenth of the unit disclosed, is also specifically disclosed. Any smaller range within the ranges disclosed or that can be derived from other endpoints disclosed are also specifically disclosed themselves. The upper and lower limits of disclosed ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art. CLAIMS

1. A method of producing a three dimensional autologous fat graft using human or animal lipoaspirate-derived adipose tissue, wherein the lipoaspirate comprises stem cells, wherein the lipoaspirate is mixed with one or more excipients, wherein the mixture of the lipoaspirate and the one or more excipients is crosslinked.

2. The method according to claim 1, wherein the one or more excipients comprise one or more biocompatible materials.

3. The method according to claim 2, wherein the one or more biocompatible materials comprise nanofibrillar cellulose.

4. The method according to claim 3, wherein the nanofibrillar cellulose is derived from tunicates, bacteria, and/or plants.

5. The method according to claim 4, wherein the nanofibrillar cellulose derived from tunicates, bacteria, and/or plants comprises a dispersion comprising fibrils with a length between 0.1 microns and 5 microns.

6. The method according to claim 4, wherein the nanofibrillar cellulose derived from tunicates, bacteria, and/or plants comprises a dispersion having a solid content greater than 1.5% and less than 4% by weight.

7. The method according to claim 4, wherein the nanofibrillar cellulose derived from tunicates, bacteria, and/or plants comprises a dispersion having an amount of hemicellulose that is lower than an amount that will substantially affect a human's immunoresponse system.

8. The method according to claim 4, wherein the nanofibrillar cellulose derived from tunicates, bacteria, and/or plants comprises a dispersion having no detectable agglomerates.

9. The method according to claim 4, wherein the nanofibrillar cellulose derived from tunicates, bacteria, and/or plants comprises a dispersion having no detectable bacteria derived lipopolysaccharides.

10. The method according to claim 4, wherein the nanofibrillar cellulose derived from tunicates, bacteria, and/or plants is crosslinked by adding (a) alginate and (b) Calcium, Barium, and/or Strontium ions.

11. The method according to claim 4, wherein the nanofibrillar cellulose derived from tunicates, bacteria, and/or plants is crosslinked by adding alginate and polylysine or other polycations.

12. The method according to claim 4, wherein the nanofibrillar cellulose derived from tunicates, bacteria, and/or plants is crosslinked by adding a solution of fibrinogen and thrombin.

13. The method according to claim 4, wherein the nanofibrillar cellulose derived from tunicates, bacteria, and/or plants is carboxymethylated or oxidized and then crosslinked by adding Calcium, Barium, and/or Strontium ions or polylysine or other polycations.

14. The method according to claim 4, wherein the nanofibrillar cellulose derived from tunicates, bacteria, and/or plants is crosslinked by adding platelet rich plasma (PRP), followed by adding Calcium ions.

15. The method according to claim 4, wherein the nanofibrillar cellulose derived from tunicates, bacteria, and/or plants is crosslinked by adding riboflavin 5′-phosphate, followed exposing to ultraviolet light.

16. An implantable composition obtained by the method according to claim 1, wherein the implantable composition is used for aesthetic and/or reconstructive surgery of a human.

17. An implantable composition obtained by the method according to claim 1, wherein the implantable composition is used for aesthetic and/or reconstructive surgery of animals.

18. An implantable composition obtained by the method according to claim 1, wherein the implantable composition is used for wound healing.

19. The method according to claim 1, wherein the three dimensional autologous fat graft is porous and survives implantation without substantial volumetric shrinkage.

20. The method according to claim 1, wherein the three dimensional autologous fat graft becomes neovascularized and is used for aesthetic and/or reconstructive surgery.
